(19) United States
(12) Patent Application Publication
(10) Pub. No.: US 2007/0020325 A1
(43) Pub. Date: Jan. 25, 2007

(54) Hydrogel composition

(75) Inventors: Mitsuru Kuribayashi, Tsukuba-shi (JP); Seiji Tokumoto, Tsukuba-shi (JP)

Correspondence Address: FITCH, EVEN, TABIN & FLANNERY, P. O. BOX 18415, WASHINGTON, DC, 20036, US

(73) Assignee: Hisamitsu Pharmaceutical Co., Inc., Tosu-shi (JP)

(21) Appl. No.: 11/490,311

(22) Filed: Jul. 21, 2006

(30) Foreign Application Priority Data
Jul. 22, 2005 (JP) P2005-213152

(51) Int. Cl. A61F 13/02; A61L 15/16

(52) U.S. Cl. 424/448

(57) ABSTRACT

A hydrogel composition containing as an active ingredient a drug that is transdermally or transmucosally absorbed to produce a drug effect, the hydrogel composition comprising polyvinyl alcohol with a saponification degree of 90-96 mol %, a water-soluble polymer and water, wherein the content of the water-soluble polymer is no greater than 25 wt % and the water content is at least 60 wt %, with respect to the total weight of the hydrogel composition.

### BACKGROUND OF THE INVENTION

1. Field of the Invention

The present invention relates to a hydrogel composition.

2. Related Background Art

Hydrogel compositions are used as formulations for transdermal or transmucosal absorption of drugs. However, conventional hydrogel compositions undergo progressive syneresis during storage forming opaque gels, and therefore fail to maintain high adhesion to the skin or mucosa.

As a hydrogel composition designed to reduce such syneresis, Japanese Patent Application Laid-Open 2001-525377 discloses a composition comprising water and a prescribed amount of polyvinyl alcohol having a prescribed hydrolysis degree. Also, Japanese Patent Publication HEI 5-80514 discloses a composition comprising polyvinylpyrrolidone with a molecular weight of 100,000-600,000, polyvinyl alcohol with a molecular weight of 150,000-300,000, a polar plasticizer (humectant) and water, with respective contents of 25-50 wt %, 2-5 wt %, 5-40 wt % and 3-50 wt %.

### SUMMARY OF THE INVENTION

However, the composition described in Japanese Patent Application Laid-Open 2001-525377 is designed to reduce syneresis, and does not necessarily provide adequately high adhesion to the skin or mucosa. Furthermore, the composition described in Japanese Patent Publication HEI 5-80514 has inadequate drug release properties because of its low moisture content, and in particular it has not exhibited sufficient drug release properties for use in iontophoresis.

It is an object of the present invention to provide a hydrogel composition comprising as an active ingredient a drug that is transdermally or transmucosally absorbed to produce a drug effect, wherein syneresis is adequately reduced, and sufficiently high adhesion to the skin or mucosa and sufficiently high drug release properties are exhibited.

In order to achieve the aforestated object, the present invention provides a hydrogel composition comprising as an active ingredient a drug that is transdermally or transmucosally absorbed to produce a drug effect, the hydrogel composition comprising polyvinyl alcohol (hereinafter also referred to as “PVA”) with a saponification degree of 90-96 mol %, a water-soluble polymer and water, wherein the content of the water-soluble polymer is no greater than 25 wt % and the water content is at least 60 wt %, with respect to the total weight of the hydrogel composition.

In the present invention, the “saponification degree” of the PVA refers to the proportion of acetyl groups of the polyvinyl acetate which are saponified to be converted to hydroxyl groups, and it is represented by the formula {p/(p+q)}×100 (mol %), where p and q stand for the numbers of moles of hydroxyl and acetyl groups, respectively, in the PVA.

The hydrogel composition comprises not conventional PVA with a saponification degree of greater than 96 mol %, but rather PVA with a relatively low saponification degree, i.e. a saponification degree of 90-96 mol %. By containing PVA with a saponification degree of 90-96 mol %, the syneresis is adequately reduced even if the water content is high (for example, 60 wt % or greater). This is believed to be because the low saponification degree of the PVA results in a looser network formed by the PVA, with water molecules being more apt to be incorporated in the network and bond with PVA, thereby reducing the free water content.

In addition, by containing PVA with a saponification degree of 90-96 mol %, the homogeneity of the formed gel has become higher. This is believed to be because the low saponification degree of the PVA allows the PVA to dissolve at a lower temperature during preparation of the gel.

The content of PVA with a saponification degree of 90-96 mol % in the hydrogel composition is preferably 5-25 wt % based on the total weight of the hydrogel composition. If the content is lower than 5 wt %, the gel strength will tend to be reduced. On the other hand, if it is greater than 25 wt %, the gel will tend to harden and to have reduced cohesion and adhesion to the skin or mucosa.

The hydrogel composition preferably also contains PVA with a saponification degree of 78-90 mol %. Containing this type of PVA will more reliably reduce syneresis.

The content of PVA with a saponification degree of 78-90 mol % is preferably no greater than 5 wt % based on the total weight of the hydrogel composition. If it is greater than 5 wt %, the gel will tend to harden and to have reduced cohesion and adhesion to the skin or mucosa.

The hydrogel composition contains a water-soluble polymer. By containing a water-soluble polymer, the cohesion and the adhesion to the skin or mucosa have become sufficiently high. The water-soluble polymer inhibits formation of a PVA network and is incorporated into the gaps in a hydrated state, thereby increasing the amount of water molecules incorporated in the network and reducing the amount of free water. Thus, containing a water-soluble polymer will further reduce syneresis.

The water-soluble polymer content is no greater than 25 wt % based on the total weight of the hydrogel composition. If it is greater than 25 wt %, phase separation may occur between the water-soluble polymer and PVA, thereby promoting syneresis.

The water-soluble polymer is preferably polyvinylpyrrolidone, which is highly compatible with PVA.

The hydrogel composition also contains water. Water bonds with the PVA and water-soluble polymer and is dispersed in the PVA network, thereby diffusing the drug and other components. Containing water therefore helps to diffuse the contained drug and other components in the gel and facilitates their release from the gel.

The water content is at least 60 wt % based on the total weight of the hydrogel composition. Containing water at 60 wt % or greater allows the contained drug to be sufficiently diffused in the gel and adequately increases the drug release properties.

The hydrogel composition preferably also contains a humectant. Containing a humectant will retain moisture at the site of attachment to the skin or mucosa. Also, since it prevents formation of a film on the surface of the starting material mixture during preparation of the gel, the homogeneity of the prepared gel is increased.

As examples of suitable humectants there may be mentioned glycerin, polyethylene glycol, propylene glycol, D-sorbitol, xylitol, mannitol, erythritol and urea. These humectants may be used alone or in combinations of two or more.

The hydrogel composition preferably also contains an electrolyte. Containing an electrolyte will prevent changes in the pH of the gel due to the drug or other components, and reduce irritation to the skin during iontophoresis.

The hydrogel composition preferably also contains a surfactant. Containing a surfactant will produce a softer gel and increase the contact area with the skin or mucosa, thereby increasing the drug release properties and drug migration onto the skin or mucosa. Also, containing a surfactant will reduce the impedance at the boundary between the gel and skin, thereby facilitating the flow of current between the gel and skin and further increasing drug migration onto the skin during iontophoresis. Bubbles produced during dissolution of the PVA also increase the impedance of the gel and lower diffusion of the drug and other components in the gel, but containing a surfactant notably inhibits generation of bubbles during dissolution of the PVA and thereby increases the drug release properties and drug migration onto the skin or mucosa. Furthermore, since it prevents formation of a film on the surface of the starting material mixture during preparation of the gel, the homogeneity of the prepared gel is increased.

The hydrogel composition contains as an active ingredient a drug that is transdermally or transmucosally absorbed to produce a drug effect. As preferred examples of such drugs there may be mentioned dexamethasone sodium phosphate, dexamethasone sodium acetate, dexamethasone sodium metasulfobenzoate, hydrocortisone sodium succinate, hydrocortisone sodium phosphate, prednisolone sodium succinate and betamethasone sodium phosphate. These drugs may be used alone or in combinations of two or more.

For gelling of a solution containing PVA with a relatively low saponification degree (96 mol % or less), it is usually necessary to carry out freezing-thawing treatment several times, but the hydrogel composition of the present invention can be prepared with a single freezing-thawing treatment by adjusting the conditions (cooling rate, temperature-elevating rate, etc.) for the freezing-thawing treatment.

Japanese Patent Application Laid-Open SHO 58-501034 discloses a composition comprising a polar plasticizer, polyvinyl alcohol and polyvinylpyrrolidone, with respective contents of about 1-60 wt %, about 6-30 wt % and about 2-30 wt %. Also, Japanese Patent Application Laid-Open SHO 62-1158744 discloses a composition comprising polyvinyl alcohol, polyvinylpyrrolidone and water, wherein the contents satisfy specified conditions and the pH is no higher than 4.5. However, these compositions do not always exhibit high adhesion to the skin or mucosa.

### DESCRIPTION OF THE PREFERRED EMBODIMENTS

Preferred embodiments of the hydrogel composition of the present invention will now be explained.

The hydrogel composition of the invention comprises PVA with a saponification degree of 90-96 mol %, a water-soluble polymer and water.

By containing PVA with a saponification degree of 90-96 mol %, the syneresis is adequately reduced even if the moisture content is high (for example, 60 wt % or greater). This is believed to be because the low saponification degree of the PVA results in a looser network formed by the PVA, with water molecules being more apt to be incorporated in the network and bond with PVA, thereby reducing the free water content.

In addition, by containing PVA with a saponification degree of 90-96 mol %, the homogeneity of the gel has become higher. This is believed to be because the low saponification degree of the PVA allows the PVA to dissolve at a lower temperature during preparation of the gel.

The content of PVA with a saponification degree of 90-96 mol % is preferably 5-25 wt % based on the total weight of the hydrogel composition. If the content is lower than 5 wt %, the gel strength will tend to be reduced. On the other hand if it is greater than 25 wt %, the gel will tend to harden and to have reduced cohesion and adhesion to the skin or mucosa.

The hydrogel composition preferably also contains PVA with a saponification degree of 78-90 mol %. Containing this type of PVA will more reliably reduce syneresis.

The content of PVA with a saponification degree of 78-90 mol % is preferably no greater than 5 wt % based on the total weight of the hydrogel composition. If it is greater than 5 wt %, the gel will tend to harden and to have reduced cohesion and adhesion to the skin or mucosa.

The PVAs with a saponification degree of 90-96 mol % and a saponification degree of 78-90 mol % both preferably have polymerization degrees of 1700-2500. A polymerization degree of lower than 1700 will tend to reduce the gel strength and will lengthen the time required for gelling. On the other hand, a polymerization degree of greater than 2500 will tend to harden the gel and to reduce its cohesion and adhesion to the skin or mucosa.

In the hydrogel composition of the invention, by containing a water-soluble polymer, the cohesion and the adhesion to the skin or mucosa have become sufficiently high. The water-soluble polymer inhibits formation of a PVA network and is incorporated into the gaps in a hydrated state, thereby increasing the amount of water molecules incorporated in the network and reducing the amount of free water. Thus, containing a water-soluble polymer will further reduce syneresis.

The water-soluble polymer content is no greater than 25 wt % based on the total weight of the hydrogel composition. If it is greater than 25 wt %, phase separation may occur between the water-soluble polymer and PVA, thereby promoting syneresis. The content of the water-soluble polymer is preferably at least 0.01 wt %. If it is less than 0.01 wt %, the shape retention of the gel and its adhesion to the skin or mucosa will tend to be reduced.

As water-soluble polymers there may be mentioned ionic compounds such as polyacrylic acid, neutralized polyacrylic acid, methoxyethylene-maleic anhydride copolymer, methoxyethylene-maleic acid copolymer, isobutylene-maleic anhydride copolymer, isobutylene-maleic acid copolymer, carboxyvinyl polymer, polyacrylamide, polyacrylamide derivatives, N-vinylacetamide, copolymers of N-vinylacetamide and acrylic acid or acrylic acid salts, carboxymethylcellulose sodium and the like. There may also be mentioned non-ionic compounds such as polyvinyl formal, polyvinylmethylether, polyvinyl methacrylate, polyvinylpyrrolidone, polyvinylpyrrolidone-vinyl acetate copolymer, polyethylene oxide, polypropylene oxide and the like. Preferred among these are polyvinylpyrrolidone and polyethylene oxide, with polyvinylpyrrolidone being particularly preferred for its high compatibility with polyvinyl alcohol.

The hydrogel composition of the invention also contains water. Water bonds with the PVA and water-soluble polymer and is dispersed in the PVA network, thereby diffusing the drug and other components. Containing water therefore helps to diffuse the contained drug and other components in the gel and facilitates their release from the gel.

Purified water is preferred as the water.

The water content is at least 60 wt % based on the total weight of the hydrogel composition. Containing water at 60 wt % or greater allows the contained drug to be sufficiently diffused in the gel and adequately increases the drug release properties. The water content is also preferably no higher than 90 wt %. A water content of greater than 90 wt % will tend to reduce the shape retention of the gel.

The hydrogel composition preferably also contains a humectant. Containing a humectant will retain moisture at the site of attachment to the skin or mucosa. Also, since it prevents formation of a film on the surface of the starting material mixture during preparation of the gel, the homogeneity of the prepared gel is increased.

The humectant content is preferably 0.1-15 wt % based on the total weight of the hydrogel composition. If it is less than 0.1 wt %, the gel will tend to lose moisture after the hydrogel composition is applied to the skin or mucosa. On the other hand, if this content is greater than 15 wt %, syneresis will tend to increase.

As humectants there may be mentioned glycols such as glycerin, ethylene glycol, diethylene glycol, triethylene glycol, polyethylene glycol, propylene glycol and polypropylene glycol, diols such as 1,3-propanediol and 1,4-butanediol, sugar alcohols such as D-sorbitol, xylitol, mannitol and erythritol, and urea. Preferred among these are glycerin, polyethylene glycol, propylene glycol, D-sorbitol, xylitol, mannitol, erythritol and urea. These humectants may be used alone or in combinations of two or more.

The hydrogel composition preferably also contains an electrolyte (where the electrolyte is not a electrolyte comprised in the drug used as an active ingredient). Containing an electrolyte will prevent changes in the pH of the gel due to the drug or other components, and reduce irritation to the skin during iontophoresis.

As electrolytes there may be mentioned sodium chloride, potassium chloride, calcium chloride, zinc chloride, aluminum chloride, ammonium chloride, calcium monohydrogenphosphate, sodium monohydrogenphosphate, potassium monohydrogenphosphate, tricalcium phosphate, trisodium phosphate, dipotassium hydrogenphosphate and disodium hydrogenphosphate.

The electrolyte content is preferably 0.001-1 wt % based on the total weight of the hydrogel composition. If it is lower than 0.001 wt %, the storage stability of the drug will tend to be reduced, and pH adjustment of the gel will be difficult. On the other hand, if it is greater than 1 wt %, interaction with a hydroxyl group of the PVA will tend to increase syneresis. The electrolyte content is more preferably 0.01-0.3 wt %.

The hydrogel composition preferably also contains a surfactant. Containing a surfactant will produce a softer gel and increase the contact area with the skin or mucosa, thereby increasing the drug release properties and drug migration onto the skin or mucosa. Also, containing a surfactant will reduce the impedance at the boundary between the gel and skin, thereby facilitating the flow of current between the gel and skin and further increasing drug migration onto the skin during iontophoresis. Bubbles produced during dissolution of the PVA also increase the impedance of the gel and lower diffusion of the drug and other components in the gel, but containing a surfactant notably inhibits generation of bubbles during dissolution of the PVA and thereby increases the drug release properties and drug migration onto the skin or mucosa. Furthermore, since it prevents formation of a film on the surface of the starting material mixture during preparation of the gel, the homogeneity of the prepared gel is increased.

The HLB (hydrophile-lipophile balance) value of the surfactant is preferably 6 or greater. If the HLB value is less than 6, the surfactant may not dissolve in the water of the gel, and the gel will tend to turn opaque. Furthermore, since ionic surfactants can adversely affect the charge balance in the gel, a non-ionic surfactant is preferred when the hydrogel composition is used for iontophoresis.

The surfactant content is preferably no greater than 2 wt % based on the total weight of the hydrogel composition. If it is greater than 2 wt %, syneresis will tend to increase.

The hydrogel composition may also contain a chelating agent.

Preferred chelating agents include ethylenediaminetetraacetic acid (EDTA) or its sodium salts (disodium edetate, etc.), potassium salts, calcium disodium salt, diammonium salt and triethanolamine salts, and hydroxyethylethylenediaminetetraacetic acid (HEDTA) or its trisodium salt.

The chelating agent content is preferably 0.001-1 wt % based on the total weight of the hydrogel composition. If it is less than 0.001 wt %, the metal ion-trapping effect will be insufficient, while if it is greater than 1 wt %, the chelating agent will tend to act as a competing ion for the drug, making it difficult for the prepared hydrogel composition to be used for iontophoresis. The chelating agent content is more preferably 0.01-0.5 wt %.

The hydrogel composition of the invention also contains as an active ingredient a drug that is transdermally or transmucosally absorbed to produce a drug effect.

So long as the contained drug is transdermally or transmucosally absorbed to produce a drug effect, it may be any selected from among antiallergic drugs, anesthetic drugs, analgesic drugs, antiasthmatic drugs, anticonvulsant drugs, antitumor drugs, antipyretic drugs, antiarrhythmic drugs, antihypertensive drugs, diuretic drugs, vasodilators, antiemetic drugs, central nervous system stimulants, diagnostic agents, hormone agents, anti-inflammatory drugs, antidepressant drugs, antipsychotic drugs, immunosuppressant drugs, muscle relaxants, antiviral drugs, antibiotics, antithrombotic drugs, bone resorption inhibitors, osteogenesis promoters and the like.

As examples of drugs which dissociate to produce a cation, there may be mentioned bacampicillin, sultamicillin, cefpodoxime proxetil, cefteram pivoxil, cefinenoxime, cefotiam, doxycycline, minocycline, tetracycline, erythromycin, rokitamycin, amikacin, arbekacin, astromicin, dibekacin, gentamicin, isepamycin, kanamycin, micronomicin, sisomicin, streptomycin, tobramycin, ethambutol, isoniazid, fluconazole, flucytosine, miconazole, acyclovir, chloramphenicol, clindamycin, fosfomycin, vancomycin, aclarubicin, bleomycin, cytarabine, dacarbazine, nimustine, peplomycin, procarbazine, vinblastine, vincristine, vindesine, calcitonins, parathyroid hormone (PTH), granulocyte colony stimulating factor (G-CSF) mecasermin, alimem azine, chlorpheniramine, clemastine, mequitazine, azelastine, ketotifen, oxatomide, methylmethionine sulfonium chloride, colchicine, camostat, gabexate, nafamostat, mizoribine, piroxicam, proglumetacin, emorfazone, tiaramide, buprenorphine, ergotamine, phenacetin, rilmazafone, triazolam, zopiclone, nitrazepam, clonazepam, amantadine, bromocriptin, chlorpromazine, sultopride, chlordiazepoxide, cloxazolam, diazepam, etizolam, oxazolam, amitriptyline, imipramine, nortriptyline, setiptiline, ticlopidine, atropine, pancuronium bromide, tizanidine, pyridostigmine bromide, dobutamine, dopamine, benidipine, diltiazem, nicardipine, verapamil, acebutolol, atenolol, carteolol, metoprolol, nipradilol, pindolol, propranolol, dipyridamole, nicorandil, trapidil, ajmaline, aprindine, dibenzoline, disopyramide, flecainide, isoprenaline, lidocaine, mexiletine, procaine, procainamide, tetracaine, dibucaine, propafenone, quinidine, hydroclilorothiazide, trichlormethiazide, tripamide, azosemide, amosulalol, budralazine, bunazosin, cadralazine, clonidine, delapril, enalapril, guanethidine, hydralazine, labetalol, prazosin, reserpine, terazosin, urapidil, nicomol, epinephrine, etilefrine, midodrine, papaverine, clenbuterol, fenoterol, mabuterol, procaterol, salbutamol, terbutaline, tulobuterol, tipepidine, ambroxol, bromhexine, cimetidine, famotidine, ranitidine, roxatidine acetate, benexate, omeprazole, pirenzepine, sulpiride, cisapride, domperidone, metoclopramide, trimebutine, codeine, morphine, fentanyl, pethidine, oxybutynin, ritodrine, terodiline, and their pharmaceutically acceptable salts.

As examples of drugs which dissociate to produce an anion, there may be mentioned amoxicillin, ampicillin, aspoxicillin, benzylpenicillin, methicillin, piperacillin, sulbenicillin, ticarcillin, cefaclor, cefadroxil, cephalexin, cefatrizine, cefixime, cefradine, cefroxadine, cefamandole, cefazolin, cefinetazole, cefminox, cefoperazone, cefotaxime, cefotetan, cefoxitin, cefpiramide, cefsulodin, ceftazidime, ceftizoxime, ceftriaxone, cefuzonam, aztreonam, carumonam, flomoxef, imipenem, latamoxef, ciprofloxacin, enoxacin, nalidixic acid, nolfroxacin, ofloxacin, vidarabine, fluorouracil, methotrexate, levothyroxine, liothyronine, amlexanox, cromoglycic acid, tranilast, gliclazide, insulin, prostaglandins, benzbromarone, carbazochrome, tranexamic acid, alclofenac, aspirin, diclofenac, ibuprofen, indomethacin, ketoprofen, mefenamic acid, sulindac, tiaprofenic acid, tolmetin, sulpymme, lobenzarit, penicillamine, amobarbital, pentobarbital, phenobarbital, thiopental, phenytoin, valproic acid, droxidopa, acetazolamide, bumetanide, canrenoic acid, ethacrynic acid, alacepril, captopril, lisinopril, methyldopa, clofibrate, pravastatin, probucol, alprostadil, aminophylline, theophylline, carbocisteine, dexamethasone phosphate, dexamethasone acetate, dexamethasone metasulfobenzoate, hydrocortisone succinate, hydrocortisone phosphate, prednisolone succinate, betamethasone phosphate, and their pharmaceutically acceptable salts.

Preferred among these drugs are dexamethasone sodium phosphate, dexamethasone sodium acetate, dexamethasone sodium metasulfobenzoate, hydrocortisone sodium succinate, hydrocortisone sodium phosphate, prednisolone sodium succinate and betamethasone sodium phosphate.

The hydrogel composition of the invention may contain a single type of drug alone, but it may also contain a combination of two or more drugs so long as no harmful drug interactions occur in the body. The drug content may be appropriately determined according to the properties of each drug

When the drug used is one with low water-solubility, it is preferred to also contain a solubilizer. Containing a solubilizer will facilitate diffusion of the drug in the gel, and thereby increase the drug release properties and drug migration onto the skin or mucosa.

As solubilizers there are preferred non-alcoholic solvents, and as examples there may be mentioned fatty acid esters such as isopropyl myristate, diisopropyl adipate, diisopropyl sebacate and oleyl oleate, animal and vegetable oils such as eucalyptus oil, squalane and squalene, as well as paraffin oils, silicone oils, N-methyl-2-pyrrolidone, crotamiton and the like. Alcoholic solvents can promote syneresis in some cases.

The type and content of the solubilizer may be appropriately determined depending on the drug used.

The hydrogel composition may further contain stabilizers, (triethanolamine, etc.), preservatives (methyl paraoxybenzoate, propyl paraoxybenzoate, etc.) and the like.

The hydrogel composition of the invention may be prepared by carrying out freezing-thawing treatment once or several times. In the freezing-thawing treatment, the aforementioned components are mixed, the mixture is cooled to freezing, and the temperature is then gradually increased for thawing.

In the freezing-thawing treatment, preferably the mixture is cooled to below −20° C. at a cooling rate of at least 0.3° C./min, allowed to stand at that temperature for 30 minutes or longer, and then heated at a temperature-elevating rate of no more than 0.3° C./min for thawing. Carrying out such treatment will allow the hydrogel composition to be prepared with sufficiently high probability by a single freezing-thawing treatment procedure. The cooling rate is more preferably at least 0.6° C./min. A cooling rate of 0.6° C./min or greater will further increase the shape retention of the prepared hydrogel composition. The cooling rate referred to here is the average cooling rate for cooling from room temperature to −20° C., and the temperature-elevating rate is the average temperature-elevating rate for heating from −10° C. to 0° C.

### EXAMPLES

The present invention will now be explained in further detail by examples and comparative examples, with the understanding that the invention is in no way limited by the examples below.

### Example 1

A hydrogel composition with the composition listed in Table 1 was prepared. The amounts of the components in Table 1 are listed as parts by weight.

First, 16.0 parts by weight of PVA with a saponification degree of 95 mol % (Kuraray Co., Ltd.), 1.5 parts by weight of PVA with a saponification degree of 89 mol % (Kuraray Co., Ltd.), 1.5 parts by weight of polyvinylpyrrolidone K90 (ISP Co.), 0.18 part by weight of methyl paraoxybenzoate, 0.02 part by weight of propyl paraoxybenzoate, 0.15 part by weight of POE(20) sorbitan monooleate and 60.45 parts by weight of purified water were mixed while heating.

Separately, 3.0 parts by weight of dexamethasone sodium phosphate, 1.0 part by weight of urea, 2.5 parts by weight of D-sorbitol solution (70%), 0.05 part by weight of sodium sulfite, 0.1 part by weight of disodium edetate, 0.05 part by weight of sodium chloride, 0.5 part by weight of triethanolamine and 13.0 parts by weight of purified water were mixed.

The mixtures were combined and the viscosity of the obtained mixture was measured using a VT-04 Viscotester (RION Co., Ltd.) at a temperature of 40° C.

A 1.0 g portion of the obtained mixture was then packed into a polyethylene terephthalate container (30 mm diameter, 1.5 mm depth) whose inner surface had been silicon-treated, and a silicon-treated polyethylene terephthalate film was attached thereto. The container was placed in a temperature-adjustable refrigerator and cooled to −20° C. at a cooling rate of 2.25° C./min, allowed to stand at that temperature for 180 minutes, and then heated to 0° C. at a temperature-elevating rate of 0.33° C./min and further warmed to room temperature to obtain a hydrogel composition.

The surface pH of the obtained hydrogel composition was measured. The surface pH measurement was conducted with an F-15 pH meter (Horiba, Ltd.) using contact glass electrodes. Three measurements were conducted for each sample, and the average was calculated.

Also, sensory testing was conducted after the sample had stood for 14 days at room temperature. In the sensory testing, adhesion to the skin or mucosa, flexibility, shape retention, releasability from the container and syneresis were evaluated. The syneresis was also evaluated after the sample had stood at 25° C. for 3 months, 6 months and 9 months.

### Examples 2-8 and Comparative Examples 1-4

Hydrogel compositions having the compositions listed in Tables 1, 2 and 3 were prepared according to Example 1. Viscosity measurement, surface pH measurement and sensory testing were carried out in the same manner as Example 1. The amounts of the components in Tables 1-3 are listed as parts by weight.

TABLE 1

| | Example 1 | Example 2 | Example 3 | Example 4 |
|---|---|---|---|---|
| Betamethasone sodium phosphate | 3 | 3 | 3 | 3 |
| Polyvinyl alcohol | 0 | 0 | 0 | 0 |
| (saponification degree: 99 mol %) | | | | |
| Polyvinyl alcohol | 16 | 16.5 | 17 | 16 |
| (saponification degree: 95 mol %) | | | | |
| Polyvinyl alcohol | 1.5 | 1 | 0.15 | 0.15 |
| (saponification degree: 89 mol %) | | | | |
| Polyvinyl pyrrolidone (K30) | 0 | 2.5 | 2.5 | 2.5 |
| Polyvinyl pyrrolidone (K90) | 1.5 | 0 | 0 | 0 |
| Carboxymethyl cellulose Na (1270) | 0 | 0 | 0 | 0.25 |
| Carboxymethyl cellulose Na (1110) | 0 | 0 | 0.25 | 0 |
| Partially neutralized polyacrylic acid | 0 | 0 | 0 | 0 |
| (NP700) | | | | |
| Glycerin | 0 | 0 | 0 | 0 |
| D-sorbitol solution (70%) | 2.5 | 2.5 | 2.5 | 2.5 |
| Urea | 1 | 1 | 1 | 1 |
| Polyethylene glycol monostearate | 0 | 0 | 0 | 0 |
| POE(20) sorbitan monooleate | 0.15 | 0.15 | 0.15 | 0.15 |
| Triethanolamine | 0.5 | 0.5 | 0.5 | 0.5 |
| Sodium sulfite | 0.05 | 0.05 | 0.05 | 0.05 |
| Sodium chloride | 0.05 | 0.05 | 0.05 | 0.05 |
| Disodium edetate | 0.1 | 0.1 | 0.1 | 0.1 |
| Methyl paraoxybenzoate | 0.18 | 0.18 | 0.18 | 0.18 |
| Propyl paraoxybenzoate | 0.02 | 0.02 | 0.02 | 0.02 |
| Purified water | 73.45 | 72.45 | 72.55 | 73.55 |

TABLE 2

| | Example 5 | Example 6 | Example 7 | Example 8 |
|---|---|---|---|---|
| Betamethasone sodium phosphate | 3 | 3 | 3 | 3 |
| Polyvinyl alcohol | 0 | 0 | 0 | 0 |
| (saponification degree: 99 mol %) | | | | |
| Polyvinyl alcohol | 16 | 16 | 16 | 18 |
| (saponification degree: 95 mol %) | | | | |
| Polyvinyl alcohol | 0 | 0 | 0 | 0 |
| (saponification degree: 89 mol %) | | | | |
| Polyvinyl pyrrolidone (K30) | 0 | 0 | 0 | 0 |
| Polyvinyl pyrrolidone (K90) | 2 | 2 | 2 | 3 |
| Carboxymethyl cellulose Na (1270) | 0 | 0 | 0 | 0 |
| Carboxymethyl cellulose Na (1110) | 0 | 0 | 0 | 0 |
| Partially neutralized polyacrylic acid | 0 | 0 | 0 | 0 |
| (NP700) | | | | |
| Glycerin | 0 | 5 | 2 | 0 |
| D-sorbitol solution (70%) | 3.5 | 0 | 0 | 0 |
| Urea | 0 | 0 | 1 | 0 |
| Polyethylene glycol monostearate | 0 | 0 | 0.2 | 0.2 |
| POE(20) sorbitan monooleate | 0.2 | 0.2 | 0 | 0 |
| Triethanolamine | 0.5 | 0.5 | 0.5 | 0 |
| Sodium sulfite | 0 | 0.05 | 0 | 0 |
| Sodium chloride | 0.05 | 0.05 | 0.05 | 0 |
| Disodium edetate | 0.1 | 0.1 | 0.1 | 0.1 |
| Methyl paraoxybenzoate | 0.18 | 0.18 | 0.18 | 0.18 |
| Propyl paraoxybenzoate | 0.02 | 0.02 | 0.02 | 0.02 |
| Purified water | 74.45 | 72.90 | 75.15 | 75.50 |

TABLE 3

| | Comp. Ex. 1 | Comp. Ex. 2 | Comp. Ex. 3 | Comp. Ex. 4 |
|---|---|---|---|---|
| Betamethasone sodium phosphate | 3 | 3 | 3 | 3 |
| Polyvinyl alcohol | 16 | 0 | 10 | 3 |
| (saponification degree: 99 mol %) | | | | |
| Polyvinyl alcohol | 0 | 16 | 0 | 3.5 |
| (saponification degree: 95 mol %) | | | | |
| Polyvinyl alcohol | 0 | 0 | 3 | 7 |
| (saponification degree: 89 mol %) | | | | |
| Polyvinyl pyrrolidone (K30) | 0 | 0 | 0 | 0 |
| Polyvinyl pyrrolidone (K90) | 0 | 0 | 3 | 0 |
| Carboxymethyl cellulose Na | 0 | 0 | 0 | 0 |
| (1270) | | | | |
| Carboxymethyl cellulose Na | 0 | 0 | 0 | 0 |
| (1110) | | | | |
| Partially neutralized polyacrylic | 0 | 0 | 0 | 0.1 |
| acid (NP700) | | | | |
| Glycerin | 2 | 2 | 10 | 10 |
| D-sorbitol solution (70%) | 0 | 0 | 0 | 0 |
| Urea | 0 | 0 | 0 | 0 |
| Polyethylene glycol monostearate | 0.2 | 0.2 | 0 | 0 |
| POE(20) sorbitan monooleate | 0 | 0 | 0 | 0 |
| Triethanolamine | 0.5 | 0.5 | 0 | 0 |
| Sodium sulfite | 0 | 0 | 0 | 0 |
| Sodium chloride | 0.05 | 0.05 | 0.05 | 0.05 |
| Disodium edetate | 0.1 | 0.1 | 0.1 | 0.1 |
| Methyl paraoxybenzoate | 0.18 | 0.18 | 0.18 | 0.18 |
| Propyl paraoxybenzoate | 0.02 | 0.02 | 0.02 | 0.02 |
| Purified water | 77.95 | 77.95 | 70.65 | 73.05 |

The results for Examples 1 to 8 and Comparative Examples 1 to 4 are shown in Tables 4 to 6. The results of the sensory testing are represented as A-D explained below.

[Adhesion]

A: Very strong adhesion; B: Strong adhesion; C: Weak adhesion; D: No adhesion.

[Flexibility]

A: Very high flexibility; B: High flexibility; C: Low flexibility; D: No flexibility.

[Shape Retention]

A: High shape retention; B: Deformed when pressed with finger, but shape retained and no crushing of gel; C: Gel relatively easily crushed;

D: Deformed with very weak force, gel crushed apart.

[Releasability]

A: No gel remaining on container; B: Trace amount of gel remaining on container; C: Small amount of gel remaining on container; D: Gel could not be released from container or gel crushed during release.

[Syneresis]

A: Little or no syneresis; B: Slightly moist to touch with finger; C: Small degree of syneresis observed; D: Significant syneresis observed.

TABLE 4

| | | Example 1 | Example 2 | Example 3 | Example 4 |
|---|---|---|---|---|---|
| Solution viscosity (cps) | | 33500 | 30500 | 34000 | 33500 |
| Surface pH | | 7.85 | 7.82 | 8.02 | 7.97 |
| Adhesion | | A | A | A | A |
| Flexibility | | A | A | A | A |
| Shape retention | | B | A | A | B |
| Releasability | | B | B | B | B |
| Syneresis | 14 days | A | A | A | A |
| | 3 months | A | A | A | A |
| | 6 months | A | A | A | A |
| | 9 months | A | A | A | A |

TABLE 5

| | | Example 5 | Example 6 | Example 7 | Example 8 |
|---|---|---|---|---|---|
| Solution viscosity (cps) | | 22500 | 30000 | 27000 | 45000 |
| Surface pH | | 7.47 | 7.69 | 8.19 | 7.75 |
| Adhesion | | A | B | A | A |
| Flexibility | | A | B | A | A |
| Shape retention | | B | A | A | A |
| Releasability | | B | A | B | B |
| Syneresis | 14 days | A | A | A | A |
| | 3 months | A | A | A | A |
| | 6 months | A | A | A | A |
| | 9 months | A | A | A | A |

TABLE 6

| | | Comp. | Comp. | Comp. | Comp. |
|---|---|---|---|---|---|
| | | Ex. 1 | Ex. 2 | Ex. 3 | Ex. 4 |
| Solution viscosity (cps) | | 15000 | 20000 | 20000 | 20000 |
| Surface pH | | 7.79 | 7.84 | 7.84 | 7.84 |
| Adhesion | | D | D | D | D |
| Flexibility | | C | B | B | B |
| Shape retention | | A | A | A | A |
| Releasability | | A | A | A | A |
| Syneresis | 14 days | A | A | B | B |
| | 3 months | C | B | D | D |
| | 6 months | D | D | D | D |
| | 9 months | D | D | D | D |

### Examples 9-13 and Comparative Examples 5-8

The same procedure was carried out as in Example 1 to prepare gels having the same composition as Example 1, but with freezing-thawing treatment carried out under the conditions (cooling rate, temperature-elevating rate, minimum temperature, time of freeze) listed in Table 7. The time of freeze referred to here is the length of time that the frozen mixture was held at a temperature of below −20° C.

TABLE 7

| | Cooling rate | Temperature-elevating | Minimum | Time |
|---|---|---|---|---|
| | (° C./min) | rate (° C./min) | temperature (° C.) | of freeze (min) |
| Example 9 | 2.32 | 0.19 | −20 | 120 |
| Example 10 | 2.50 | 0.21 | −20 | 120 |
| Example 11 | 0.36 | 0.21 | −20 | 120 |
| Example 12 | 1.50 | 0.17 | −36 | 96 |
| Example 13 | 0.83 | 0.19 | −37 | 84 |
| Comp. Ex. 5 | 2.32 | 2.00 | −20 | 120 |
| Comp. Ex. 6 | 0.36 | 2.22 | −20 | 120 |
| Comp. Ex. 7 | 5.00 | 0.56 | −40 | 114 |
| Comp. Ex. 8 | 7.50 | 0.56 | −38 | 204 |

No gels formed in Comparative Examples 5 to 8, but satisfactory gels were obtained in Examples 9 to 13. The gel obtained in Example 11 had slightly lower shape retention compared to the gels obtained in Examples 9, 10, 12 and 13.

According to the present invention, there is provided a hydrogel composition comprising as an active ingredient a drug that is transdermally or transmucosally absorbed to produce a drug effect, wherein syneresis is adequately reduced, and sufficiently high adhesion to the skin or mucosa and sufficiently high drug release properties are exhibited.

The hydrogel composition of the present invention can be used for development of novel formulations for transdermal/transmucosal drug administration.

What is claimed is:

1. A hydrogel composition containing as an active ingredient a drug that is transdermally or transmucosally absorbed to produce a drug effect,
the hydrogel composition comprising polyvinyl alcohol with a saponification degree of 90-96 mol %, a water-soluble polymer and water,
wherein the content of the water-soluble polymer is no greater than 25 wt % and the water content is at least 60 wt %, with respect to the total weight of the hydrogel composition.

2. The hydrogel composition according to claim 1, wherein the content of the polyvinyl alcohol with a saponification degree of 90-96 mol % is 5-25 wt % with respect to the total weight of the hydrogel composition.

3. The hydrogel composition according to claim 1, which further comprises polyvinyl alcohol with a saponification degree of 78-90 mol %.

4. The hydrogel composition according to claim 2, which further comprises polyvinyl alcohol with a saponification degree of 78-90 mol %.

5. The hydrogel composition according to claim 3, wherein the content of the polyvinyl alcohol with a saponification degree of 78-90 mol % is no greater than 5 wt % with respect to the total weight of the hydrogel composition.

6. The hydrogel composition according to claim 4, wherein the content of the polyvinyl alcohol with a saponification degree of 78-90 mol % is no greater than 5 wt % with respect to the total weight of the hydrogel composition.

7. The hydrogel composition according to claim 1, wherein said water-soluble polymer is polyvinylpyrrolidone.

8. The hydrogel composition according to claim 1, which further comprises a humectant.

9. The hydrogel composition according to claim 8, wherein said humectant is at least one selected from the group consisting of glycerin, polyethylene glycol, propylene glycol, D-sorbitol, xylitol, mannitol, erythritol and urea.

10. The hydrogel composition according to claim 1, which further comprises an electrolyte.

11. The hydrogel composition according to claim 1, which further comprises a surfactant.

12. The hydrogel composition according to claim 1, wherein said drug is at least one selected from the group consisting of dexamethasone sodium phosphate, dexamethasone sodium acetate, dexamethasone sodium metasulfobenzoate, hydrocortisone sodium succinate, hydrocortisone sodium phosphate, prednisolone sodium succinate and betamethasone sodium phosphate.
